(19) United States
(12) Patent Application Publication
(10) Pub. No.: US 2025/0276112 A1
(43) Pub. Date: Sep. 4, 2025

(54) ARTIFICIAL BONE IMPLANTS WITH NANOSCAFFOLD FOR SELF-TRIGGERED OSTEOGENIC DIFFERENTIATION OF STEM CELLS

(71) Applicant: City University of Hong Kong, Hong Kong (HK)

(72) Inventors: Jinlian HU, Hong Kong (HK); Hanbai WU, Hong Kong (HK); Shuo SHI, Hong Kong (HK)

(21) Appl. No.: 18/592,571

(22) Filed: Mar. 1, 2024

(51) Int. Cl. A61L 27/44; A61F 2/28; A61L 27/38; A61L 27/54; A61L 27/56; C08L 27/16

(52) U.S. Cl. CPC A61L 27/44; A61F 2/28; A61L 27/3834; A61L 27/54; A61L 27/56; C08L 27/16; A61L 2430/02

(57) ABSTRACT

An artificial bone implant designed to support stem cell differentiation for enhanced osteogenesis is provided. The implant features a nanostructured scaffold, including a nanofiber mat with diameters ranging from 150 nm to 300 nm, arranged either in an aligned or randomly-distributed configuration. The nanofibers, predominantly polyvinylidene fluoride (PVDF), incorporate bioactive agents to promote the growth and viability of bone marrow-derived mesenchymal stem cells (BMSCs) and osteoblasts. Following annealing polarization, the nanofibers exhibit an electroactive β-phase, constituting a significant percentage of the nanofiber composition. Plasma treatment renders the nanofibers partially hydrophilic. Notably, the electroactivity of the nanofiber scaffold plays a pivotal role in facilitating the osteogenic differentiation of bone marrow-derived mesenchymal stem cells.

### FIELD OF THE INVENTION

The present invention generally relates to the fields of biotechnology and regenerative medicine. More specifically the present invention relates to an artificial bone implant with nanostructured scaffold for facilitating stem cell differentiation.

### BACKGROUND OF THE INVENTION

Bone, viewed as a natural piezoelectrical material in engineering terms, undergoes piezoelectric effects during normal physiological activities such as bone formation, repair, and remodeling. Osteocytes, originating from bone marrow-derived mesenchymal stem cells (BMSCs), act as mechanosensory cells responding to externally stimulated cell deformation. These cells play a role in actively and passively modifying the microenvironment, including secretion and ion exchange.

Understanding changes in the cellular microenvironment and how piezoelectric materials regulate mesenchymal stem cell fate is critical for bone tissue repair and regeneration. The spontaneous generation of potentials during cell behaviors like adhesion and migration on piezoelectric materials can provide non-invasive electrical stimulation, influencing stem cell differentiation. For instance, piezoelectric materials have proven instrumental in directing stem cell fate, orchestrating processes such as directional differentiation, maintenance of stemness, and controlled proliferation. In particular, electrical signals, emanating from cell adhesion, activate receptors on focal adherent stem cells, facilitating the precise regulation of differentiation. Another noteworthy example is the piezoelectric material PLLA, characterized by robust piezoelectric properties, which autonomously generates electrical signals, significantly amplifying the proliferation of neural stem cells while preserving their stemness. Critically, the neural differentiation capacity and functionality of these cells, propelled by local electrical expansion through piezoelectricity, remain unimpaired. Moreover, studies have demonstrated the ability of piezoelectric effects to induce the in-situ aggregation and uniform distribution of stem cells on nanofiber surfaces, further highlighting the versatile applications of piezoelectric materials in stem cell regulation.

However, the dynamic response of cell behaviors in this process is not extensively reported, and the mechanisms behind differentiation promotion and regeneration through intrinsic on-demand electrical stimulation remain incompletely explored.

Currently, electrical stimulation has been verified to change the membrane potential and trigger voltage-gate calcium channels (VGCC), allowing extracellular calcium ions to flow into cells through calcium ion channels and causing calcium transform. High extracellular calcium ion concentrations have been shown to enhance the osteogenesis of human BMSCs and propagate calcium ion signals when they differentiate into osteoblasts, and active calcium signaling is involved in signal transduction to enhance bone formation. These calcium ions activate calmodulin and consume ATP increasingly. Moreover, electrostimulation directly enhances ATP synthesis for the reorganization of G-actin depolymerized from F-actin to the novel F-actin. F-actin remodeling indicates the potential osteogenic differentiation of stem cells. Despite these findings, few studies have delved into the dynamic changes between F-actin and microenvironment, let alone the adhesive area change of BMSCs related to osteogenesis on PVDF nanofibers.

Therefore, the present invention aims to address these gaps by providing a nanostructured scaffold facilitating stem cell differentiation, promotion, and regeneration through intrinsic on-demand electrical stimulation, particularly in the context of PVDF nanofibers, while dynamically monitoring calcium signals during stem cell differentiation.

### SUMMARY OF THE INVENTION

It is an objective of the present invention to provide material, or method to solve the aforementioned technical problems.

In accordance with a first aspect of the present invention, an artificial bone implant is provided. Specifically, the artificial bone implant has a nanostructure scaffold for supporting stem cell differentiation, including a nanofiber mat, the nanofibers having nanofiber diameters ranging from 150 nm to 300 nm, the nanofibers being in an aligned configuration or in a randomly-distributed configuration. The nanofibers are polyvinylidene fluoride (PVDF) nanofibers, and include one or more bioactive agents to benefit the growth and survival of bone marrow-derived mesenchymal stem cells (BMSCs) and/or osteoblasts. The PVDF nanofibers have undergone an annealing polarization such that the PVDF nanofiber includes an electroactive β-phase of at least 70 percent of the nanofibers. Furthermore, the PVDF nanofibers have undergone a plasma treatment to render the nanofibers at least partially hydrophilic. It is worth noting that the PVDF nanofiber scaffold facilitates bone marrow-derived mesenchymal stem cell osteogenic differentiation due to its electroactivity.

In accordance with one embodiment of the present invention, the aligned configuration possesses a better piezoelectrical properties than the randomly-distributed configuration.

In accordance with one embodiment of the present invention, the randomly-distributed configuration provides an increased cell contact area to the BMSCs and facilitates the calcium influx.

In accordance with another embodiment of the present invention, the artificial bone implant dynamically adjusts the calcium ion transmission of the BMSCs to provide a microenvironment suitable for cell growth.

In accordance with one embodiment of the present invention, the artificial bone implant yields piezoelectrical voltages to the BMSCs.

In accordance with one embodiment of the present invention, the annealing polarization is conducted at a temperature ranging from 70° C. to 120° C. for a duration of 4 hour to 6 hours.

In accordance with a second aspect of the present invention, a method for fabricating the artificial bone implant is introduced. Particularly, the method includes:
preparing a pre-polymer PVDF solution;
electrospinning the pre-polymer solution with a voltage of 20 kV-25 kV, a syringe pump flow rate of 0.1 ml/h-0.5 ml/h, and a receiving distance of 10 cm-15 cm to generate nanofibers on a collector and form a PVDF nanofiber mat;
subjecting the PVDF nanofiber mat to an annealing polarization so as to form an electroactive-phase of at least 70 percent of the PVDF nanofibers; and
subjecting the PVDF nanofiber mat to a plasma treatment to render the PVDF nanofibers at least partially hydrophilic.

In accordance with one embodiment of the present invention, the PVDF nanofibers have a diameter ranging from 150 nm to 300 nm.

In accordance with another embodiment of the present invention, the pre-polymer solution is prepared by dissolving 15-25% (wt.) of PVDF and 0.1-0.3% (wt.) of lithium chloride into 10-25% (wt.) of dimethylformamide and acetone solvent.

In accordance with one embodiment of the present invention, the pre-polymer PVDF solution further includes a bioactive agent.

In accordance with one embodiment of the present invention, the artificial bone implant yields piezoelectrical voltages to the BMSCs so as to adjusts the calcium ion transmission of the BMSCs to provide a microenvironment suitable for cell growth.

In accordance with a third aspect of the present invention, a method for manufacturing a bone graft for a subject in need is provided. Specifically, the method includes:
culturing autologous BMSCs of the subject on the aforementioned
artificial bone implant to obtain a bone graft; and
grafting the bone graft to a bone damaged area in the subject.

In accordance with one embodiment of the present invention, the method further includes coating the artificial bone implant with a bioactive material before culturing the autologous BMSCs to enhance cell adhesion and proliferation.

In accordance with a fourth aspect of the present invention, a living bone implant, including autologous BMSCs and the aforementioned artificial bone implant, is introduced. Particularly, the autologous BMSCs are grated onto the artificial bone implant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIG. 1 depicts a schematic diagram showing the fabrication of annealed aligned and random distributed polyvinylidene fluoride (PVDF) nanofiber mat of the artificial bone implant, and the mechanism of BMSCs differentiation on nanofibers;

FIGS. 2A-2H depict the morphology and characterization of PVDF nanofibers of the nanostructure scaffold, in which FIG. 2A shows the scanning electron microscope (SEM) images of aligned annealed PVDF nanofiber film (AA), FIG. 2B displays the SEM images of randomly distributed annealed PVDF nanofiber film (RA), FIG. 2C demonstrates the strain-stress curves of annealed and non-annealed PVDF nanofiber film, FIG. 2D depicts the broken elongation and strength of PVDF-based films, FIG. 2E demonstrates the X-ray diffraction (XRD) spectra of the random nanofibers annealed at different temperatures from 70 to 120° C., FIG. 2F depicts the XRD spectra of the aligned nanofibers annealed at different temperatures from 70 to 120° C., FIG. 2G exhibits the Fourier-transform infrared spectroscopy (FT-IR) spectrum of random nanofibers annealed at different temperatures, and FIG. 2H shows the FT-IR spectrum of the aligned nanofibers annealed at different temperatures;

FIGS. 3A-3G show the piezoelectrical properties of the artificial bone implant, in which FIG. 3A depicts the variation of the β-phase fraction with different temperature-treated AA and RA, FIG. 3B displays the piezoelectric coefficient d33 change for AA and RA with annealed at different temperatures, FIG. 3C exhibits the atomic force microscope (AFM) image of PVDF nanofibers, FIG. 3D depicts the piezo-response force microscopy (PFM) amplitude difference image of PVDF nanofibers, FIG. 3E shows the amplitude and phase hysteresis loops of random nanofibers, FIG. 3F shows the output voltage and peak voltages of the random nanofibers under 0.5 N external force and different annealing temperatures, and FIG. 3G depicts the output voltage of the aligned nanofibers treated with different temperatures under external force of 0.5 N;

FIGS. 4A-4G depict the qualitative and quantitative analysis of cells attached to the artificial bone implant, in which FIG. 4A shows the water contact angle of the PVDF-based film before and after oxygen plasma treatment, FIG. 4B depicts the cell proliferation testing of non-annealed and annealed PVDF membranes on day 1, day 3, and day 5, FIG. 4C shows the cytotoxicity assay of BMSCs seeded on non-annealed and annealed nanofibers, FIG. 4D displays the fluorescence images of BMSCs cultured on tissue culture dish (TCD), AA, and RA, where nucleus is stained in blue, actin is stained in green, and vinculin is stained in red, FIG. 4E demonstrates the nucleus area of BMSCs on TCD, AA, and RA, and FIG. 4F and FIG. 4G respectively show the distribution of cellular and actin angles on AA and RA;

FIGS. 5A-5H show the osteogenic differentiation of BMSCs on AA and RA, in which FIG. 5A depicts the Alizarin red s (ARS) staining of BMSCs on 6-well plates, RN, and RA, FIG. 5B displays the mineralized area of ARS staining of BMSCs on 6-well plates, RN, and RA, FIG. 5C shows the immunofluorescent staining of Runx2, OCN, and COL-1 of BMSCs cultured on TCD, AA, and RA after 7 days of incubation, FIG. 5D exhibits the heatmap of osteogenic-related gene expression among different samples during BMSCs differentiation, and FIGS. 5E-5H demonstrate the relative expression of osteogenic-related genes COL-1 (FIG. 5E), ALP (FIG. 5F), OCN (FIG. 5G), and RUNX2 (FIG. 5H) of BMSCs seeded on TCD, AN, RN, AA, and RA;

FIG. 6 displays that the nanofibers of the artificial bone implant are collapsed due to cell focal adhesion;

FIGS. 7A-7D depict the mechanism of better osteogenesis and calcium variation on RA than AA, in which FIG. 7A is a conceptual graphic of cells on RA having more adhesive area and more active calcium ions transfer, FIG. 7B displays the focal adhesion area of cells on TCD, AA, and RA, FIG. 7C depicts the SEM images of cells attached on TCD, AA, and RA, and FIG. 7D depicts that the cells attached to AA recalled the pseudopods in less than 2 min;

FIGS. 8A-8E depict the quantification of active calcium signals in BMSCs cultured with TCD (FIG. 8A), RN (FIG. 8B), AN (FIG. 8C), AA (FIG. 8D), and RA (FIG. 8E), respectively;

FIG. 9 depicts the single peak of the calcium signal in BMSCs cultured with the artificial bone implant;

FIG. 10 depicts the real-time calcium signals of BMSCs cultured on annealed aligned nanofibers, where the white circles mark inactive calcium signals, red circles indicate active calcium signals and yellow circles show discovered calcium signal; and

FIG. 11 depicts the responsive activity and time of intracellular calcium ions of BMSCs seeded on random nanofibers, where the white circles mark inactive calcium signals, red circles indicate active calcium signals and yellow circles show discovered calcium signal.

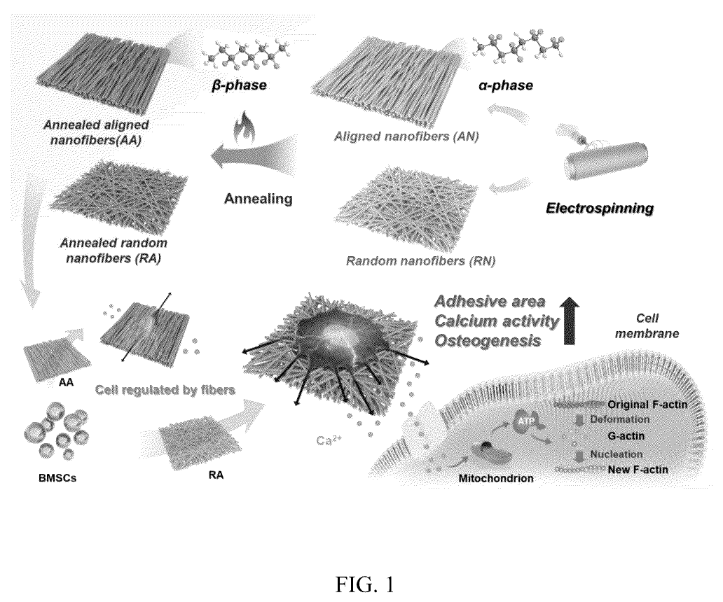

### DETAILED DESCRIPTION

In the following description, materials, methods, and/or applications of artificial bone implants for facilitating bone marrow-derived mesenchymal stem cell osteogenic differentiation and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, an artificial bone implant is provided. The artificial bone implant is designed to foster optimal conditions for stem cell differentiation, particularly bone marrow-derived mesenchymal stem cells (BMSCs) and osteoblasts.

The implant includes a nanostructured scaffold, primarily featuring a nanofiber mat characterized by nanofiber diameters ranging from 150 nm to 300 nm. The nanofibers are strategically arranged in either an aligned or randomly-distributed configuration to influence subsequent cellular interactions.

These nanofibers, primarily composed of polyvinylidene fluoride (PVDF), are not merely structural components but incorporate one or more bioactive agents. These agents play a crucial role in supporting the growth and survival of BMSCs and osteoblasts, thereby enhancing the implant's efficacy. To ensure heightened electroactivity, a key attribute for osteogenic differentiation, the nanofibers undergo an annealing polarization process. This treatment results in the induction of an electroactive β-phase, constituting 70 percent of the nanofiber composition. Additionally, a plasma treatment is employed to render the nanofibers at least partially hydrophilic, contributing to enhanced cellular interactions.

The configuration of the nanofiber scaffold holds paramount importance in influencing its properties. In one embodiment, the aligned configuration is demonstrated to possess superior piezoelectrical properties compared to the randomly-distributed configuration. This is particularly advantageous for eliciting robust responses in BMSCs. Conversely, the randomly-distributed configuration offers increased cell contact area to BMSCs, thereby facilitating calcium influx, a critical factor in bone biology. It is worth noting that the artificial bone implant dynamically adjusts the calcium ion transmission of the BMSCs to provide a microenvironment suitable for cell growth.

As used herein, the term “randomly-distributed configuration” refers to a configuration in which the individual nanofibers, formed through the electrospinning process using PVDF, exhibit no specific pattern or alignment. In this context, the nanofibers are deposited in a haphazard manner, lacking a consistent orientation or direction. The term implies that the nanofibers are dispersed chaotically, with no predominant order, allowing them to cross, intertwine, or align in various directions. This randomness in the distribution of PVDF nanofibers can result in a three-dimensional network of fibers with diverse orientations, providing unique properties to the resulting material, such as increased surface area and enhanced mechanical characteristics.

As used herein, the term “aligned configuration” refers to a configuration in which the individual nanofibers, produced through the electrospinning process using PVDF, exhibit a specific and consistent orientation in the same direction. Unlike randomly distributed nanofibers, where there is no predominant order, aligned nanofibers are intentionally organized to follow a particular axis. Furthermore, directional nanofiber patterns enable directional diffusion of cells. This alignment can be achieved through the manipulation of the electrospinning process, where external forces or collector design are used to guide the fibers in a uniform direction.

The artificial bone implant, owing to its design and treatment processes, generates piezoelectrical voltages that contribute to the dynamic interplay with BMSCs. The annealing polarization, conducted at temperatures ranging from 70° C. to 120° C. for a duration of 4 to 6 hours, ensures optimal electroactive properties. This careful balance of structural design and treatment methods establishes an artificial bone implant that not only provides structural support but actively contributes to and promotes bone regeneration through enhanced osteogenic differentiation of BMSCs.

In accordance with a second aspect of the present invention, a method for fabricating the aforementioned artificial bone implant is provided. The method begins with the preparation of a pre-polymer PVDF solution. This solution is crafted by dissolving 15-25% (wt.) of PVDF and 0.1-0.3% (wt.) of lithium chloride into 10-25% (wt.) of dimethylformamide and acetone solvent. The resulting pre-polymer PVDF solution serves as the foundation for the subsequent electrospinning process.

Electrospinning is employed to transform the pre-polymer PVDF solution into a nanofiber mat. This process involves applying a voltage in the range of 20 kV-25 kV, a syringe pump flow rate of 0.1 ml/h-0.5 ml/h, and maintaining a receiving distance of 10 cm-15 cm to ensure optimal nanofiber formation on a collector. The resulting PVDF nanofiber mat serves as the structural basis for the artificial bone implant.

Following the electrospinning process, the nanofiber mat undergoes an

annealing polarization treatment. This crucial step is essential for inducing the formation of an electroactive β-phase within the nanofibers. The annealing process is carefully executed at temperatures conducive to achieving this electroactive state, ensuring that at least 70 percent of the nanofibers exhibit this desired β-phase.

Subsequently, the nanofiber mat undergoes a plasma treatment. This treatment is applied with precision to render the nanofibers at least partially hydrophilic. The plasma treatment enhances the biocompatibility of the nanofibers and improves the ability of cell attachment, promoting favorable interactions with biological components.

Moreover, the nanofibers produced through this method have diameters ranging from 150 nm to 300 nm, a critical parameter for optimal performance. Additionally, the pre-polymer PVDF solution used in this process can be further enriched with a bioactive agent. This inclusion ensures that the resulting nanofiber mat not only provides structural support but also incorporates therapeutic elements to benefit the growth and survival of BMSCs and osteoblasts.

Due to the piezoelectrical voltages yielded by the artificial bone implant, the artificial bone implant is able to adjust the calcium ion transmission of the BMSCs to provide a better microenvironment for the BMSCs.

In accordance with a third aspect of the present invention, a method for manufacturing a bone graft for a subject in need is demonstrated. This method involves a strategic cultivation process where autologous bone marrow-derived mesenchymal stem cells from the subject are cultured directly on the aforementioned artificial bone implant. The result is a personalized crafted bone graft that encapsulates the unique electroactive features of the artificial bone implant, designed to foster and augment osteogenic differentiation in the cultured cells.

A pivotal step in this manufacturing process is the subsequent grafting of the cultivated bone graft onto a bone-damaged area within the subject. This grafting procedure seamlessly integrates the customized bone graft into the targeted region, harnessing the regenerative potential of the artificial bone implant. The implant's electroactivity, stemming from the specific nanofiber configuration and thermal annealing polarization treatment, plays a crucial role in promoting cell differentiation and enhancing the overall efficacy of the bone graft.

As an additional enhancement to this method, a coating step is introduced, where the artificial bone implant is coated with a bioactive material before initiating the culture of autologous bone marrow-derived mesenchymal stem cells. This coating serves to augment cell adhesion and proliferation, further optimizing the interaction between the implant and the cultured cells.

In accordance with a fourth aspect of the present invention, a living bone implant is provided. This implant integrates autologous BMSCs with the previously described artificial bone implant. The implant design involves the grafting of autologous BMSCs onto the substrate of the artificial bone implant.

To enact this process, autologous BMSCs are carefully isolated from the patient's own bone marrow, ensuring compatibility and minimizing the risk of immunological rejection. The living bone implant, thus created, signifies a harmonious amalgamation of the patient's own regenerative potential with the electroactive and bioactive features inherent in the artificial bone implant.

The grafted autologous BMSCs adhere to the nanofiber scaffold with an aligned or randomly distributed configuration, facilitating a seamless integration into the three-dimensional microenvironment provided by the nanofiber mat. The electroactivity of the artificial bone implant serves as a stimulus, influencing the behavior and differentiation of the grafted BMSCs.

This living bone implant not only provides structural support akin to traditional implants but also serves as a dynamic platform for cellular activities. The amalgamation of autologous BMSCs and the artificial bone implant establishes a symbiotic relationship, wherein the biological elements respond to and interact with the electroactive nanofibers. This reciprocal relationship enhances the overall performance of the living bone implant, fostering a conducive environment for cell proliferation, differentiation, and ultimately contributing to effective bone regeneration.

The utilization of autologous BMSCs in conjunction with the artificial bone implant is a significant advancement in the field, offering a personalized and regenerative approach to bone tissue engineering.

### EXAMPLES

### Cell Culture and Differentiation

BMSCs (iCell Bioscience Inc) are cultured in minimum essential medium α (MEM α) with 10% fetal bovine serum and 1% penicillin-streptomycin solution in an incubator with humidified 5% CO₂ atmosphere at 37° C. Cells are seeded on both aligned and random PVDF nanofiber mats under incubating conditions for 48 hours, and differentiated into conditional MEM a. The medium for cell culture is changed every two days when mineralized nodules are formed.

### In Vitro Analysis of Cells on PVDF Nanofiber

Cell proliferation on PVDF nanofiber is determined by cell counting kit-8 assay. Cytotoxicity of normal, non-annealing, and annealing nanofibers is measured by Live/Dead assay kit. Cells have been imaged with confocal microscope. To investigate the focal adhesion of cells, bisbenzimide (Hoechst 33342), filamentous actin tracking stain, anti-vinculin antibody, and goat anti-mouse IgG H&L are used to stain the cells. The angle of the cell and actin, size of the nucleus, and area of vinculin are quantitatively analyzed using image software (ImageJ).

### Example 1. Fabrication and Characterization of PVDF Nanofibers of an Artificial Bone Implant

The electrospinning process is employed to fabricate aligned annealed PVDF nanofiber membranes/films (AA) and randomly distributed annealed PVDF nanofiber membranes/films (RA) of the artificial bone implant, each collected under different collecting speeds. Briefly, pre-polymer solutions are prepared by dissolving 20% (wt.) of PVDF and 0.2% (wt.) of lithium chloride into 15% (wt.) of dimethylformamide and acetone. The pre-polymer solution is pumped at 0.3 ml/h, with a needle-to-collector distance of 15 cm and a voltage of 20 kV applied. The average nanofiber diameter is determined using image software (ImageJ). The crystallinity of the electrospun membranes is enhanced through annealing for 6hours at different temperatures, for instance, the PVDF films under different temperatures in oven for 6 hours. For improved cell attachment, the nanofibers undergo a 1.5-minute plasma cleaning system for 1.5 min.

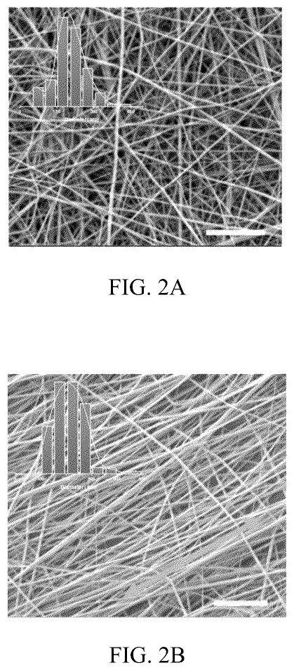

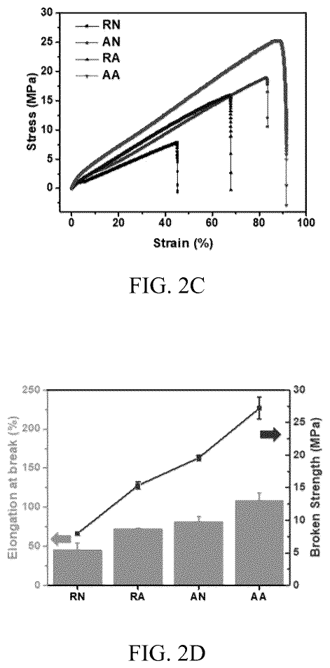

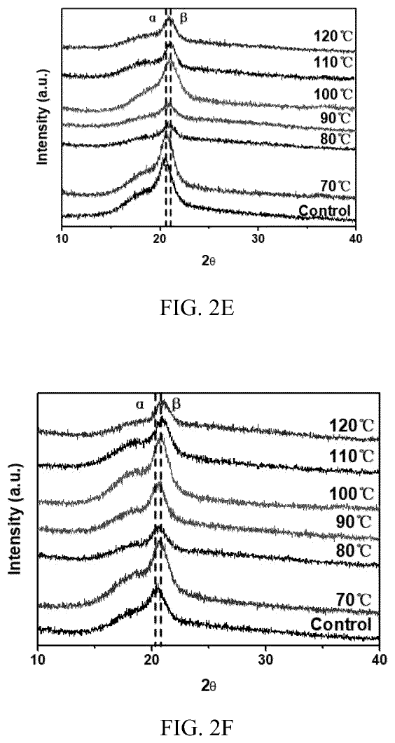

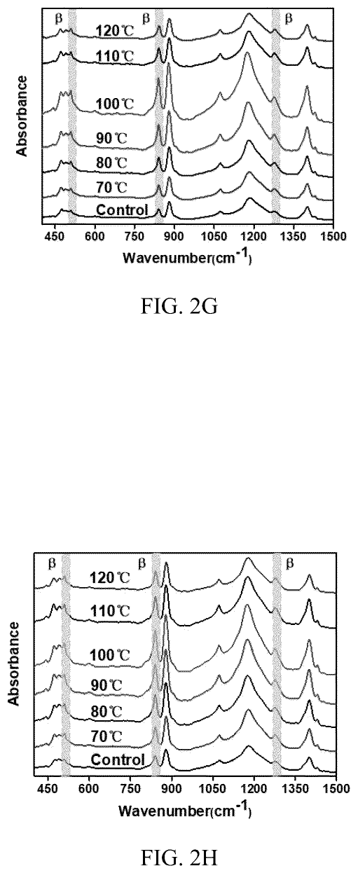

The morphology, orientation, and mechanical properties of nanofibers in the artificial bone implant play crucial roles in the piezoelectric effect. Therefore, the distribution, crystallinity, and piezoelectrical voltage of nanofibers are measured. The topography of PVDF nanofibers is characterized by scanning electron microscope (SEM). As shown in FIGS. 2A-2B, the SEM images and the diameters of the PVDF nanofibers of both AA and RA PVDF nanofiber films reveal that they display highly ordered and disordered, respectively. In addition, PVDF nanofibers ranged from 75-500 nm, and most of them are concentrated at 150 to 300 nm. No significant difference in nanofiber diameters is observed among randomly distributed PVDF nanofiber film (RN), aligned PVDF nanofiber film (AN), RA, and AA. To further verify the orientation of nanofibers, mechanical properties, and elongation at break are evaluated. The tensile strength (FIG. 2C) of AN and AA are two times better than RN and RA, respectively. RA and AA are heat treated under 100° C. before testing. The broken strength and elongation at break (FIG. 2D) show that AA contains the best mechanical properties with a 108% elongation rate and a higher broken strength at 27.17 MPa. AN and AA exhibit better mechanical properties and greater elongation than RN and RA because of the highly ordered structures.

Fourier transform infrared (FT-IR) spectrometer is utilized to analyze the spectrums from 400 to 4000 cm⁻¹. The crystallinity of PVDF nanofibers is analyzed with X-ray diffraction system in order to examine the formation of β-phase in PVDF. As shown in FIG. 2E and FIG. 2F, generally, the peak at 20.1° corresponds to 110reflections of the monoclinic α-phase crystal, and the peak at 20.6° corresponds to 110/200 reflections of the orthorhombic β-phase. Different crystalline phases of

PVDF are identified by the FT-IR spectra (FIG. 2G and FIG. 2H). The characteristic peaks at 530, 766, 855, 976, and 1423 cm⁻¹ are α-phase, and peaks at 509, 840, and 1275 cm⁻¹ are attributed to β-phase. The XRD and FT-IR results indicate that the β-phase peak is increased with temperature, and the highest peak is measured at 100° C.

### Example 2. Evaluation of the Piezoelectrical Properties of the Artificial Bone Implant

The β-phase of PVDF is renowned for its exceptional electroactive properties among all phases, and an elevated β-phase content in PVDF enhances the piezoelectricity of materials. The relevant fraction of the β-phase (F(β)) is calculated by eliminating the absorbance of α-and β-phases, determined using Equation (1).

$$F ( β ) = \frac{{A}_{β}}{1.3 \times {A}_{α} + {A}_{β}} ; \qquad ( 1 )$$

where A_{α} and A_{β} are the absorbencies at 764 cm⁻¹ and 840 cm⁻¹, respectively.

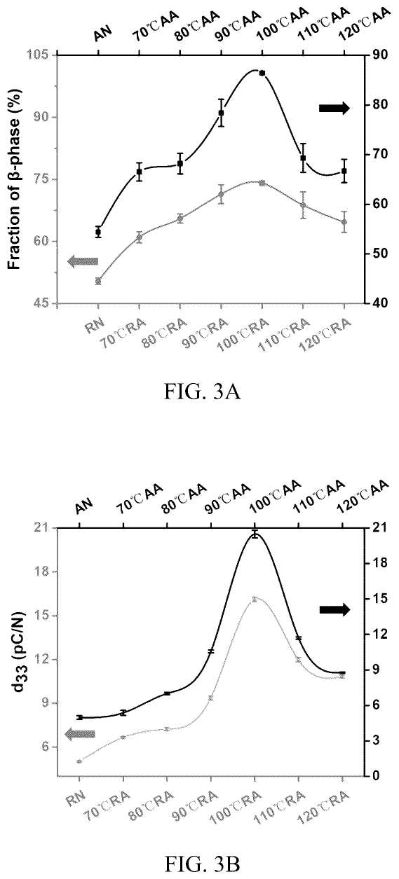

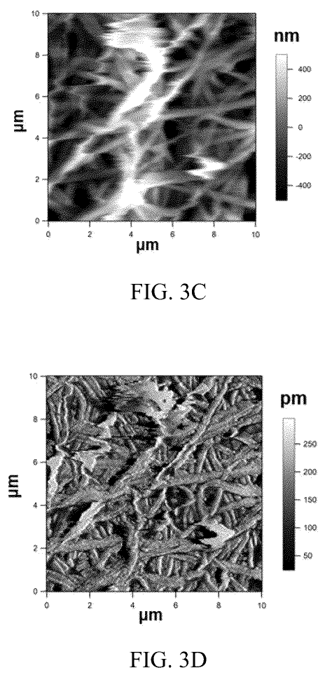

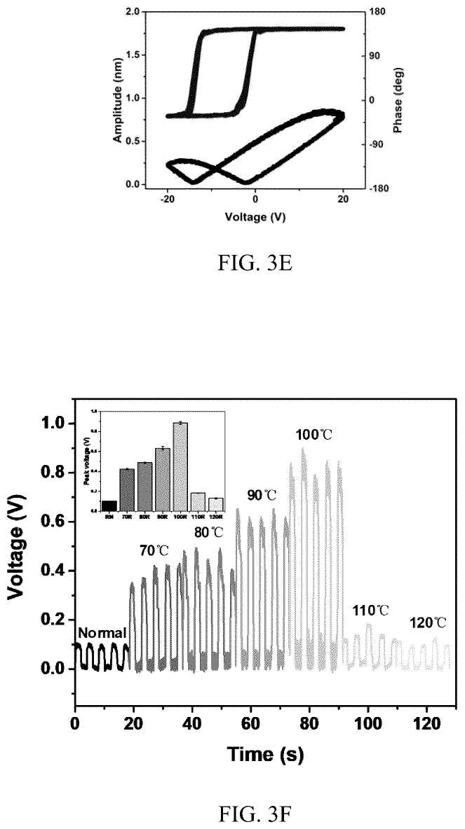

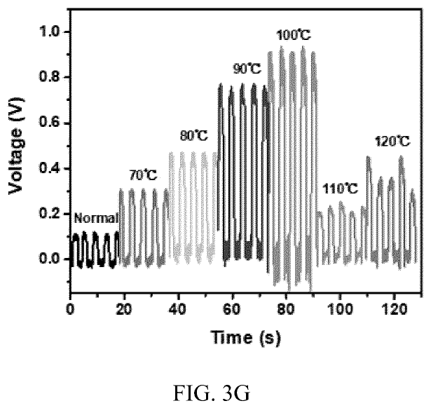

Both AA and RA exhibit increased β-phase contents with higher annealing temperatures (FIG. 3A), reaching high β-phase fractions of 86.45% and 74.14% at 100° C., respectively. In contrast, pure PVDF membranes only contain a β-phase proportion ranging from 50% to 85%.

The average piezoelectric coefficient d₃₃ (FIG. 3B) exhibits an upward trend with temperature, measuring 16.1 and 20.5 pC/N for AA and RA, respectively. Compared to untreated PVDF nanofibers, the d₃₃ of annealed PVDF nanofibers increases by at least four times. To further validate the piezoelectrical properties, piezo-response force microscopy (PFM) is employed. The random structure of nanofibers is observed through atomic force microscope (AFM) in FIG. 3C, consistent with the SEM image results (FIG. 2A). Quantitative and qualitative assessments of the amplitude and phase change produced by random nanofibers are presented in FIG. 3D and FIG. 3E. The results demonstrate at least 200 pm amplitudes under a 2V bias with butterfly-shaped loops and 180 degrees switching hysteresis loops under a 20 V bias, respectively. Additionally, charges generated by the piezoelectric effect induce endogenous cell potentials, promoting bone differentiation or regeneration.

The output voltage of the nanofiber membranes generated via the piezoelectric effect is recorded separately (FIG. 3F and FIG. 3G). Aluminum tapes are applied to both sides of the film as electrodes before measurement. Under a stable external force (0.5 N), the output voltage for sensitive nanofibers gradually increases from 0.12 V to 0.94 V, for RA it is from 0.1 V to 0.9 V at 100°° C., and rapidly decreases when the temperature exceeds 110° C. The results indicate that AA exhibits higher piezoelectrical properties than RA due to better polarization. Since AA and RA achieve an excellent piezoelectric effect under 100° C. annealing treatment, this temperature is used to prepare the annealed samples for further experiments.

### Example 3. Biocompatibility and Biological Cues of Cells Seeded on the Annealed Membranes

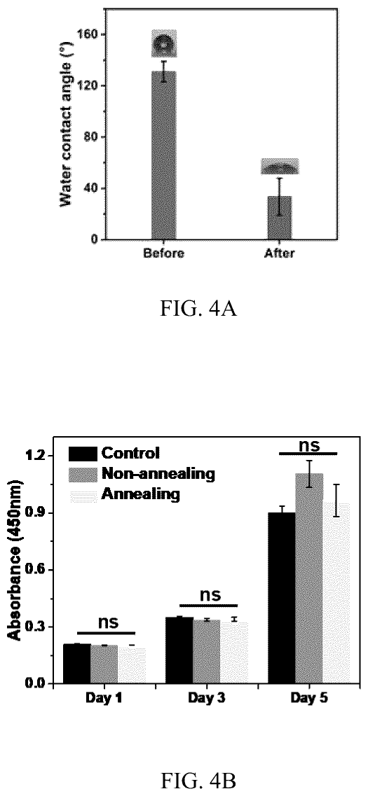

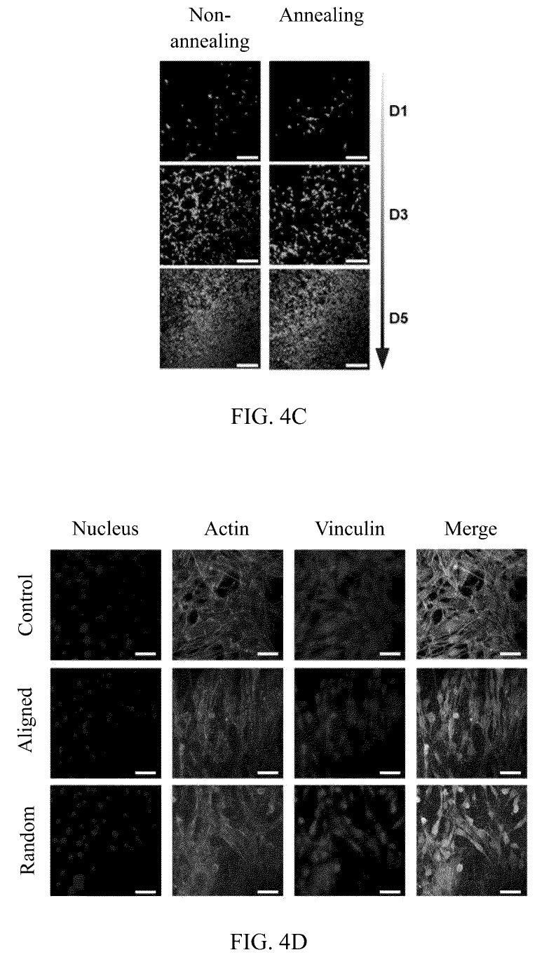

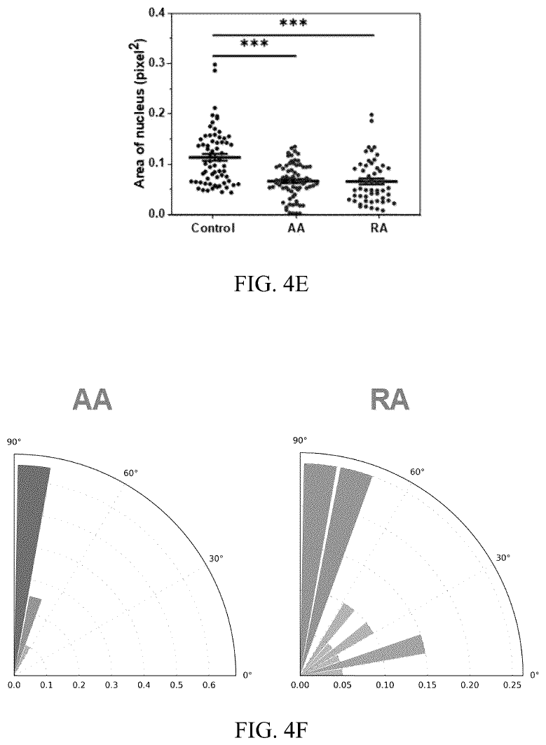

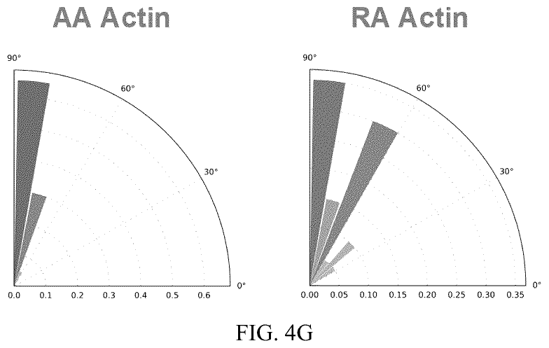

Leveraging their outstanding piezoelectrical properties, a comprehensive assessment of the biological attributes of the prepared PVDF-based films is conducted. To examine the cytotoxicity of the annealing treatment, focal adhesion of cells, and the orientation of cell attachment, BMSCs are cultured with PVDF membranes for further experimentation. Hydrophobic PVDF membranes undergo oxygen-plasma treatment to enhance cell attachment, resulting in a substantial decrease in the water contact angle from 129.6° to 20.7° (FIG. 4A). The investigation into cell proliferation ratio and survival rate involves co-culturing cells with RN and RA for 5 days, with cells seeded on tissue culture dishes (TCDs) serving as controls. The rapid increase in the proliferation ratio (FIG. 4B) signifies robust cellular activity. Furthermore, Live/Dead staining images reveal no apparent dead cells (FIG. 4C), underscoring the commendable biocompatibility of PVDF nanofibers both before and after annealing.

To delve into the cell attachment status to PVDF nanofibers, the nucleus, F-actin, and vinculin of BMSCs incubated with PVDF nanofibers are characterized using a confocal microscope. Fluorescence images (FIG. 4D) demonstrate that BMSCs on TCD exhibit a well-spread cytoskeleton.

Remodeling of the cytoskeleton and compression of the nucleus indicate the potential for differentiation induced by PVDF nanofibers. Therefore, the area of nuclei is thoroughly investigated. Quantitative analysis of nuclei area (FIG. 4E) reveals that the size of the nucleus on TCD is larger than that on AA and RA. However, there are no significant overall differences in nuclei size of cells cultured on electrospun networks. Cells on both AA and RA exhibit differentiation potential. In FIG. 4D, the angle of nanofibers is predominantly distributed at approximately 90°. Most angles of cells and actin also approach 90° (FIG. 4F). In contrast, cells dispersed on random nanofibers demonstrate both a random orientation of cells and their actin, ranging from 0° to 90° (FIG. 4G). Notably, cells and their actin align and parallel the orientation of AA. The F-actin of cells on nanofibers is stretched and rearranged in comparison with cells seeded on TCD.

### Example 4. Osteogenic Ability of Cells on the Annealed PVDF Membranes

As the potential stem cell differential induction showed by PVDF membranes, the osteogenic ability is evaluated by immunofluorescence (IF) staining, reverse transcription polymerase chain reaction (RT-PCR), and Alizarin red s (ARS) staining. Briefly, BMSCs are cultured in a conditional differentiation medium for 7-21 days. All cells are fixed before testing in 4% paraformaldehyde solution and washed with phosphate-buffered saline. Fixed cells are permeabilized with 0.1% Triton X-100 before immunofluorescence staining. Permeabilized cells are washed by PBS. The primary antibodies anti-COLIA antibody, osteocalcin antibody, and Runx2 Antibody and secondary antibody, fluorescent-labeled goat anti-mouse IgG, are used after blocking at 37° C. with goat serum. The fluorescence images are captured using the confocal microscope. ARS staining kit is used to demonstrate the mineralization of cells seeded on different nanofibers. Stained samples are washed with DI water and observed under an inverted microscope (TS100, Nikon). ARS-stained area (%) of samples is additionally evaluated by image analyzing software. For RT-PCR, BMSCs are cultured on the TCD, AN, RN, AA, and RA for 14 days as described previously. The total RNA of BMSCs seeded on the TCD and PVDF nanofibers are extracted. RNA was measured using a Nanodrop to detect the concentration. An equivalent amount of RNA is transcribed into complementary DNA (cDNA) according to the instructions of a reverse transcription kit. Total cDNA samples are subsequently stored at −20° C. Then the qPCR reaction for osteogenic genes is performed using SYBR Green and a RT-PCR detection system. Quantification of target genes is evaluated by 2-ΔΔ CT method. The ΔCt value of each sample is calculated by comparing it with the Ct values of a housekeeping gene, beta-actin.

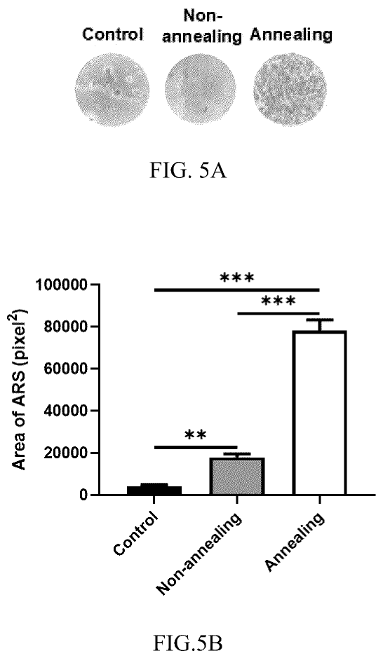

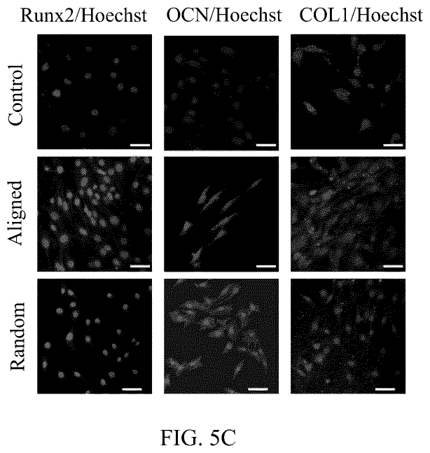

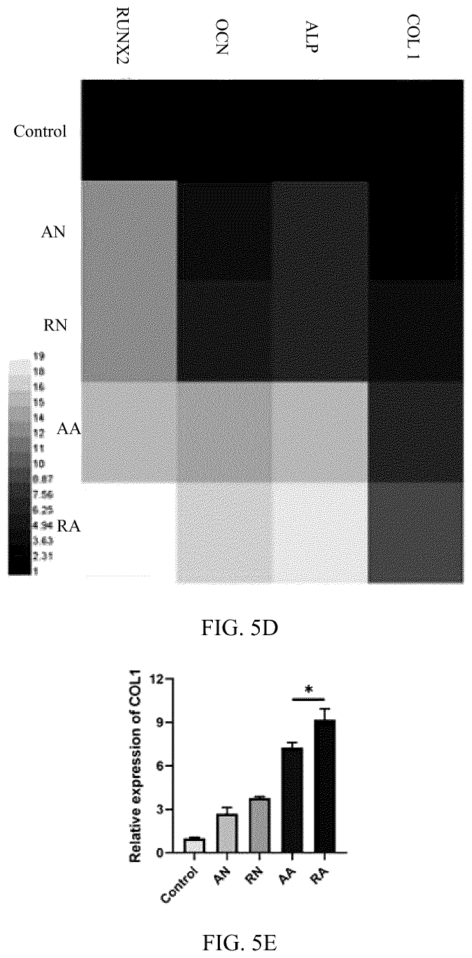

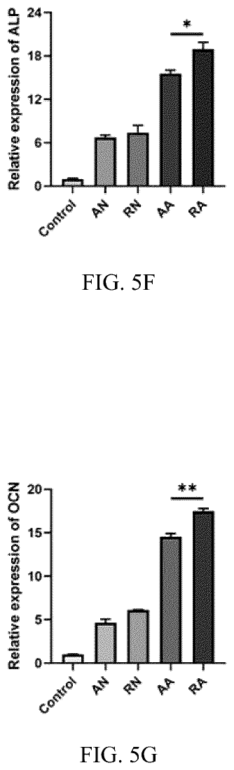

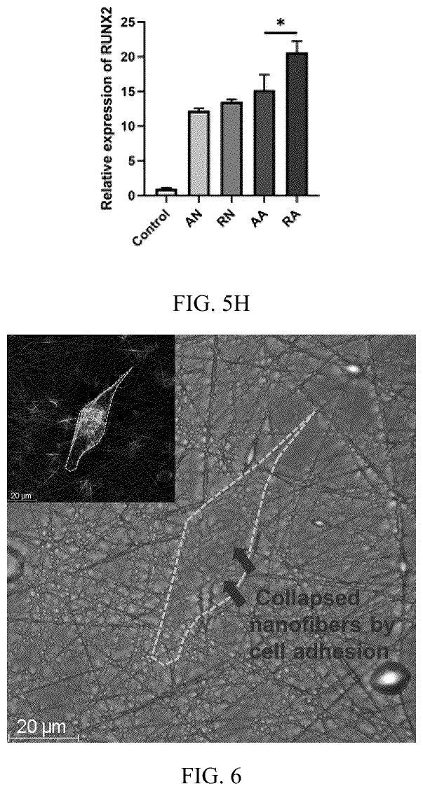

In order to verify that if the mineralization of BMSCs is mostly induced by annealed PVDF membranes, the mineralizing bone nodules of cells differentiated on TCD, RN, and RA are evaluated by ARS staining (FIG. 5A). Quantitative analysis of the mineralized area indicates that non-annealed PVDF nanofiber film improves the mineralization of BMSCs. However, annealed PVDF nanofiber film presents 4-fold mineralization compared with non-annealed one (FIG. 5B). The major regulator Runt-related transcription factor 2 (Runx2) of osteogenesis, osteocalcin (OCN) that manages bone remodeling, and the primary organic component of bone extracellular matrix collagen type I (COL-1) are used to detect the osteogenesis for BMSCs seeded on TCD, AA and RA incubated with PVDF nanofibers. The fluorescence intensity of cells on AA and RA is stronger than that on TCD (FIG. 5C). However, IF staining of cells on AA and RA is indistinguishable. Therefore, the relative expression of the osteogenic genes alkaline phosphatase (ALP), OCN, and COL-1 is determined via RT-PCR after 21 days of cultivation. Based on the results of RT-PCR, the relationship between different gene expression levels and samples is directly displayed in the heatmap. This indicates that osteogenesis of BMSCs on RA is better than that on AA (FIG. 5D). It is also found that the targeted gene expression of ALP, OCN, COL-1, and RUNX2 in RA and AA is significantly two to three times higher than that in RN and AN (FIGS. 5E-5H). Therefore, RA and AA have significant differences in that osteogenesis of cells in RA is better than in AA.

### Example 5. Investigations in Mechanism and Calcium Signal Determination

The present invention posits that the piezoelectrical effect of the artificial bone implant promotes calcium ion transmission for it's both RA and AA, and the random structure of RA provides the best-growing microenvironment for BMSCs. Calcium ions play an important role in bone biology. Simultaneously, a greater spreading area of cells on the artificial bone implant leads to more adhesion and creates more opportunities for intercellular communication.

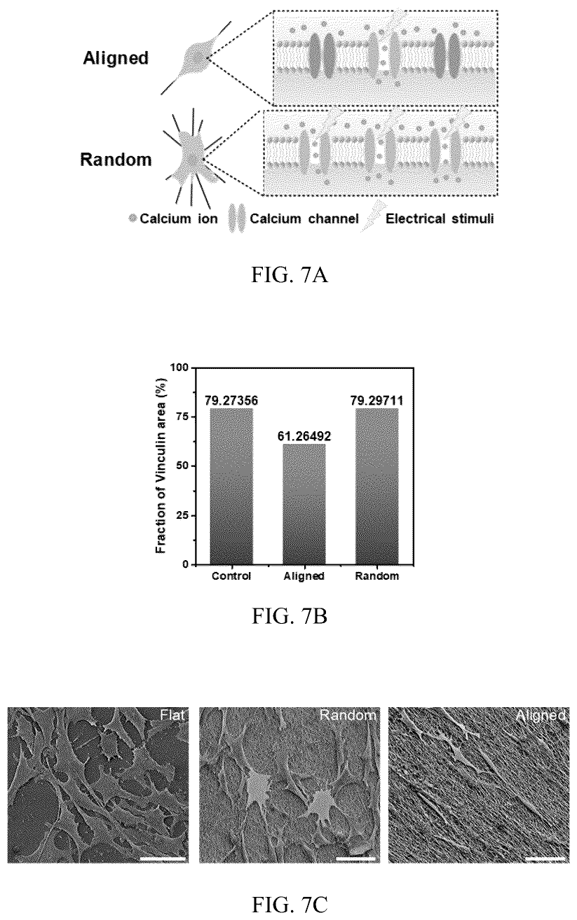

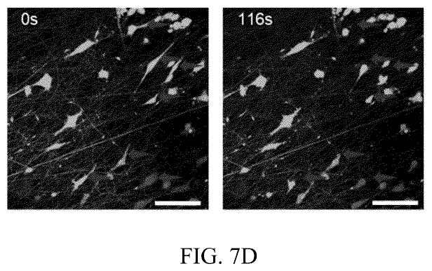

In terms of osteogenesis, RA can adjust the stem cell fate more effectively than AA. Cells dispersed on AA and RA are stretched by electrospun networks, and cell adhesion causes the deformation of nanofibers (FIG. 6). The mechanism of BMSCs osteogenic differentiation on RA is further investigated (FIG. 7A). More adhesive area leads to more active calcium transfer. The cellular focal adhesion area of vinculin is investigated in FIG. 7B. BMSCs seeded on TCD and random nanofibers show close fractions at 79.27% and 79.30%, respectively. Additionally, based on the SEM images of cells seeded on different substrates (FIG. 7C), cells irregularly attach to TCD. Cells attached to TCD and RA present a flatter shape, while cells on AA have more pseudopodia that are regulated by aligned nanofibers. However, BMSCs cultured on aligned nanofibers show a shuttle shape and pseudopodia on two sides of cells dragged by nanofibers. Nevertheless, nanofibers not only provide the 3D microenvironment for cell attachment but also stretch the cells and regulate them. However, BMSCs cultured on AA only present a 61.26% vinculin area. This further proves that the cells attach to more area on random nanofibers than aligned ones, and the cells are more flattened. The alignment of nanofibers regulates the cells, implying less adhesive area and fewer connecting points. Furthermore, it is found that the cells recall some pseudopods from the AA (FIG. 7D) in 2 min. This is also decisive evidence proving that the cells need more adhesive points on random nanofibers for stable cell behaviors.

To confirm that the cells on random nanofibers have healthier cell behaviors, the calcium indicator is used to label the calcium activity of cells on TCD, RN, AN, RA, and AA. In brief, the cells are cultured on samples for 24 hours and incubated with 2.28 μM Fluo-4 AM for 1 hour at 37° C. The redundant dye is removed by PBS and washed three times. The concentration of calcium ions is determined every 2 seconds during 1200 seconds with the confocal microscope, and the signals are analyzed using ImageJ.

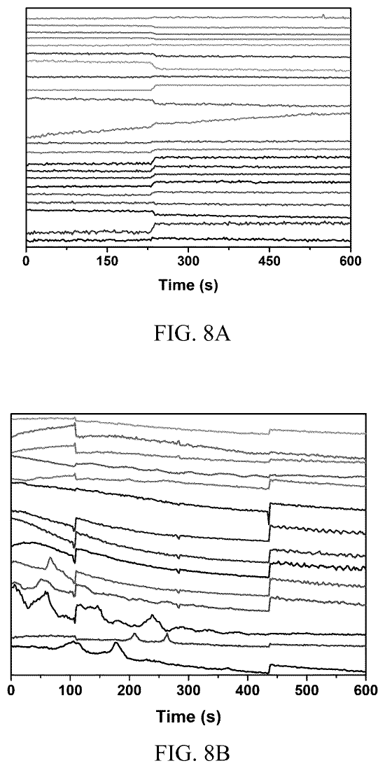

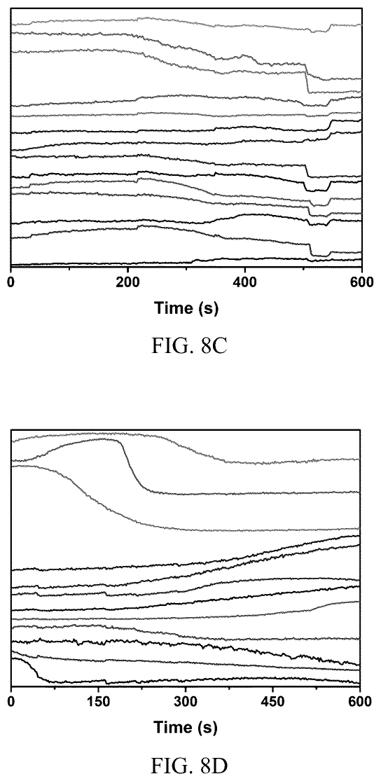

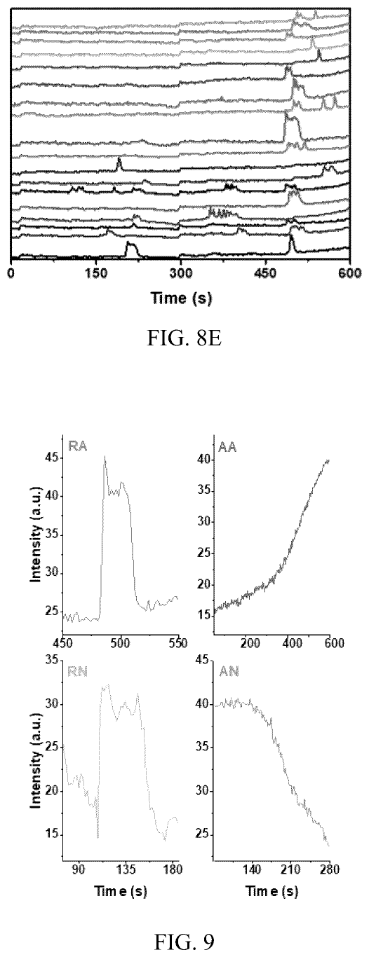

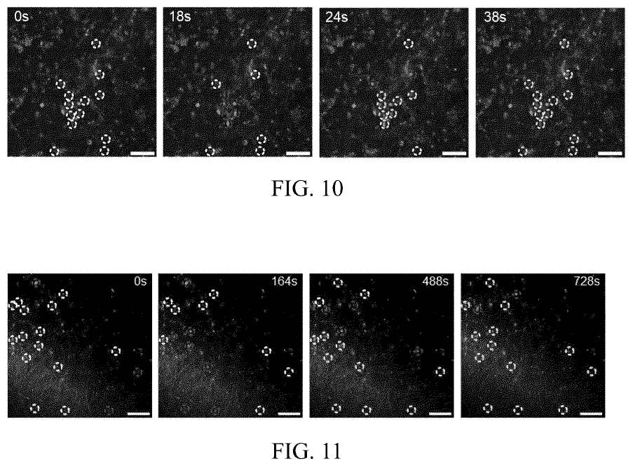

As shown in FIGS. 8A-8E, the active calcium signals are quantitatively detected on TCD, AN, RN, AA, and RA. The single peak of the calcium signal is also analyzed (FIG. 9), and the calcium signals on RA and RN show a similar pulse signal in 40 s. However, the signals on AA and AN spend 7 to 17 times more seconds. In addition, the intensity of signals on RA and AA is 10 a.u. stronger than those on RN and AN. These results evidence that piezoelectrical voltages synergistically improve cell activity. During the 600 s dynamic monitoring, the calcium signals of cells attached to TCD as a control show no evident change. Nevertheless, calcium activity is identified in BMSCs incubated with RA and AA. Calcium signals presented in cells attached to the RA are ephemeral but vibrant. For cells cultivated with AA, calcium signals are sluggish. Intracellular calcium ions are transported fast on RA in 38 seconds (FIG. 10). However, the calcium ions inside cells are gradually transformed on AA in 728 seconds (FIG. 11), which is almost 20 times slower than that on RA.

In summary, two different oriented PVDF nanofibers with excellent properties are provided as a film for facilitating osteogenic ability of stem cells. The PVDF nanofiber film can be annealed under an annealing temperature ranging from 70° C. to 120° C. for significantly increasing the β-phase content (86.45% and 74.14%, respectively) compared to most polarized pure PVDF films. These PVDF membranes arrange the stem cell fate through a great piezoelectrical effect and more active calcium transfer, BMSCs seeded on RA nanofibers exhibits a larger vinculin area, improved osteogenic potential, and active calcium signaling even without the need for external stimulation. The BMSCs on both AA and RA exhibit greater osteogenesis than that on TCD. This is caused by the piezoelectrical effect triggered by cells on high β-phase content PVDF membranes. In addition, cells on RA have more adhesion area, stable microenvironment, and active calcium variation which promote the better differentiation of cells on RA than AA. The comprehensive analysis conducted in the present invention provides a fresh perspective for investigating the intricate interactions within the microenvironment. Moreover, these findings lay down the fundamental principles for the design of next-generation scaffolds aimed at bone repair.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. An artificial bone implant, comprising:
a nanostructure scaffold for supporting stem cell differentiation, including a PVDF nanofiber mat, the PVDF nanofibers having nanofiber diameters ranging from 150 nm to 300 nm, the PVDF nanofibers being in an aligned configuration or in a randomly-distributed configuration;
the PVDF nanofibers including one or more bioactive agents to benefit the growth and survival of bone marrow-derived mesenchymal stem cells (BMSCs) and/or osteoblasts;
the PVDF nanofibers having undergone an annealing polarization such that the nanofibers include an electroactive β-phase of at least 70 percent of the PVDF nanofibers;
the PVDF nanofibers having undergone a plasma treatment to render the nanofibers at least partially hydrophilic and improve the ability of cell attachment;
wherein, the nanostructure scaffold facilitates bone marrow-derived mesenchymal stem cell osteogenic differentiation due to its electroactivity.

2. The artificial bone implant of claim 1, wherein the aligned configuration possesses a better piezoelectrical properties than the randomly-distributed configuration.

3. The artificial bone implant of claim 1, wherein the randomly-distributed configuration provides an increased cell contact area to the BMSCs and facilitates the calcium influx.

4. The artificial bone implant of claim 3, wherein the artificial bone implant dynamically adjusts the calcium ion transmission of the BMSCs to provide a microenvironment suitable for cell growth.

5. The artificial bone implant of claim 1, wherein the artificial bone implant yields piezoelectrical voltages to the BMSCs.

6. The artificial bone implant of claim 1, wherein the annealing polarization is conducted at a temperature ranging from 70° C. to 120° C. for a duration of 4 hour to 6 hours.

7. A method for fabricating the artificial bone implant of claim 1, comprising:
preparing a pre-polymer PVDF solution;
electrospinning the pre-polymer PVDF solution with a voltage of 20 kV-25 kV, a syringe pump flow rate of 0.1 ml/h-0.5 ml/h, and a receiving distance of 10 cm-15 cm to generate PVDF nanofibers on a collector and form a PVDF nanofiber mat;
subjecting the PVDF nanofiber mat to an annealing polarization so as to form an electroactive β-phase of at least 70 percent of the PVDF nanofibers; and
subjecting the PVDF nanofiber mat to a plasma treatment to render the nanofibers at least partially hydrophilic and improve the ability of cell attachment.

8. The method of claim 7, wherein the nanofibers have a diameter ranging from 150 nm to 300 nm.

9. The method of claim 7, wherein the pre-polymer PVDF solution is prepared by dissolving 15-25% (wt.) of PVDF and 0.1-0.3% (wt.) of lithium chloride into 10-25% (wt.) of dimethylformamide and acetone solvent.

10. The method of claim 9, wherein the pre-polymer PVDF solution further comprises a bioactive agent.

11. The method of claim 7, wherein the artificial bone implant yields piezoelectrical voltages to the BMSCs so as to adjusts the calcium ion transmission of the BMSCs to provide a microenvironment suitable for cell growth.

12. A method for manufacturing a bone graft for a subject in need, comprising:
culturing autologous BMSCs of the subject on the artificial bone implant of claim 1 to obtain a bone graft; and
grafting the bone graft to a bone damaged area in the subject.

13. The method of claim 12, further comprising coating the artificial bone implant with a bioactive material before culturing the autologous BMSCs to enhance cell adhesion and proliferation.

14. A living bone implant, comprising autologous BMSCs and the artificial bone implant of claim 1, wherein the autologous BMSCs are grafted onto the artificial bone implant.
